(19) United States
(12) Patent Application Publication
(10) Pub. No.: US 2012/0046569 A1
(43) Pub. Date: Feb. 23, 2012

(54) METHOD AND APPARATUS

(75) Inventors: Stuart John Johnstone, Balgownie (AU); Steven James Roodenrys, Keiraville (AU)

(73) Assignee: UNIVERSITY OF WOLLONGONG, Wollongong, New South Wales (AU)

(21) Appl. No.: 13/255,839

(22) PCT Filed: Mar. 9, 2010
(86) PCT No.: PCT/AU10/00260

§ 371 Date: Oct. 13, 2011

(30) Foreign Application Priority Data
Mar. 11, 2009 (AU) 2009901053

(51) Int. Cl. A61B 5/0484; G09B 19/00

(52) U.S. Cl. 600/544; 434/247

(57) ABSTRACT

A method of providing cognitive training to a user, the method including, in a processing system, presenting a cognitive task to a user, the cognitive task requiring the user to view presented information and provide at least one input response; determining the at least one input response using an input device; determining from a measuring device a measure of electrical activity in the user's brain whilst performing the cognitive task; and, determining a score based on at least one of the at least one input response and the measured electrical activity.

### BACKGROUND OF THE INVENTION

The present invention relates to a method and apparatus for providing cognitive training to a user, and in particular to a method and apparatus that involves determining a measure of electrical activity in the user's brain.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

WO2008/091323 describes a noise-free portable EEG system including hardware and software that can quantitatively evaluate mental state. The quantitative data of mental states and their levels can be applied to various areas of brain-machine interface including consumer products, video game, toys, military and aerospace as well as biofeedback or neurofeedback.

In the case of games, or other similar arrangements, a measure of electrical activity determined by the EEG system is used to control an aspect of the game. Thus, for example, the user can be required to control their brain wave patterns to thereby control the position of a cursor on a screen. Whilst this can assist with improving the user's control over brain wave patterns, this does not necessarily assist in developing important cognitive skills concurrently with attention and concentration. Additionally, the user's ability to control their brain waves often relies on the direct feedback provided by the game, rendering this of little use in day-today situations.

### SUMMARY OF THE PRESENT INVENTION

The present invention seeks to substantially overcome, or at least ameliorate, one or more disadvantages of existing arrangements.

In a first broad form the present invention provides a method of providing cognitive training to a user, the method including, in a processing system:
a) presenting a cognitive task to a user, the cognitive task requiring the user to view presented information and provide at least one input response;
b) determining the at least one input response using an input device;
c) determining from a measuring device a measure of electrical activity in the user's brain whilst performing the cognitive task; and,
d) determining a score based on at least one of the at least one input response and the measured electrical activity.

Typically the cognitive task includes a number of difficulty levels, and wherein the method includes, in the processing system, selecting a difficulty level for the task at least partially in accordance with the score from a previous cognitive task.

Typically the method includes, in the processing system, selecting a difficulty level for the cognitive task in accordance with a score indicative of a success measure of a previous cognitive task.

Typically the method includes, in the processing system:
a) determining a first score using the at least one input response;
b) determining a second score using the measured electrical activity; and,
c) determining the score using the first and second scores.

Typically the method includes, in the processing system:
a) comparing the input response to a required response; and,
b) determining the first score in accordance with results of the comparison.

Typically the method includes, in the processing system:
a) determining an electrical activity indicator using the measured electrical activity;
b) comparing the electrical activity indicator to indicator criteria; and,
c) determining a second score in accordance with results of the comparison.

Typically the electrical activity indicator is indicative of at least one characteristic of brain function.

Typically the electrical activity indicator is at least partially indicative of at least one of:
a) attention; and,
b) focus.

Typically the method includes, in the processing system:
a) determining a baseline electrical activity indicator using the measured electrical activity prior to performing a cognitive task; and,
b) determining indicator criteria at least partially in accordance with the baseline electrical activity indicator.

Typically the task is at least one of:
a) an inhibition task;
b) a memory task; and,
c) spatial working memory task.

Typically the method includes, in the processing system:
a) presenting a sequence of representations to the user, each representation being associated with a respective required response;
b) for each representation, comparing at least one input response to the respective required response; and,
c) determining a score at least partially in accordance with results of the comparison.

Typically the at least one required response associated with at least one of the representations is a null response.

Typically the method includes, in the processing system:
a) selecting one of a plurality of objects to be a target object;
b) presenting representations of the plurality of objects to the user;
c) determining user selection of an object in accordance with an input response;
d) determining if the user selected object is the target object; and,
e) determining a score at least partially in accordance with results of the determination.

Typically the score is used to assess cognitive function.

Typically the score is used as an incentive to improve cognitive function.

In a second broad form the present invention provides apparatus for providing cognitive training to a user, the apparatus including a processing system for:
a) presenting a cognitive task to a user, the cognitive task requiring the user to view presented information and provide at least one input response via an input device;
b) receiving from a measuring device a measure of electrical activity in the user's brain whilst performing the cognitive task; and,
c) determining a score based on at least one of the at least one input response and the measured electrical activity.

Typically the apparatus includes the measuring device.

### BRIEF DESCRIPTION OF THE DRAWINGS

An example of the present invention will now be described with reference to the accompanying drawings, in which:

FIG. 1 shows a schematic diagram of an example of apparatus for providing cognitive training to a user;

FIG. 2 is a flow chart of an example of a method for operating providing cognitive training;

FIGS. 3A and 3B are flow charts of a second example of a method for operating providing cognitive training;

FIG. 4 is a flow chart of an example of an inhibition task; and,

FIG. 5 is a flow chart of an example of a working memory task.

### DETAILED DESCRIPTION OF THE PREFERRED EMBODIMENTS

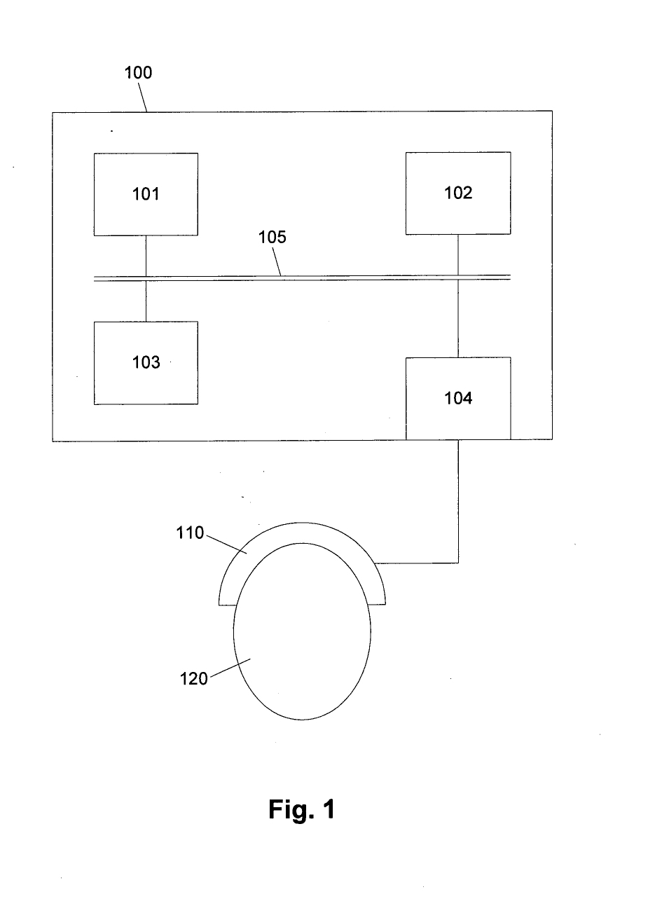

An example of apparatus for providing cognitive training will now be described with reference to FIG. 1.

In one example, the apparatus includes a computer system 100, coupled to a measuring device 110, which in use is connected to, or worn on, a user's head 120 to allow a measure of electrical activity in the user's brain to be measured.

The measure of electrical brain activity can be of any suitable form, but in one example is at least partially indicative of an attention or meditation level of the user. This is typically based at least partially on the level of beta brain wave activity in the user's brain, but may also depend to a lesser degree on other brain wave activity, such as gamma brain waves, or the like.

The measuring device 110 can therefore be of any suitable form that is able to determine information regarding relevant brain activity, and in one example can be similar to the measuring device described in WO2008/09132. The measuring device 110 therefore typically includes a non-invasive, dry, bio-sensor that measures neurological activity and optionally muscle movements, and generates corresponding electrical signals. The signals may be provided directly to the computer system 100, or at least partially processed in processing electronics integrated into the measuring device 110, before being transferred to the computer system 100.

In use, the computer system 100 is adapted to present cognitive tasks to the user, and then interpret signals received from the measuring device 110. Accordingly, the computer system may be of any suitable form.

In one example, the computer system includes a processor 101, a memory 102, an input/output device 103, such as a keyboard and display; and an external interface 104, coupled together via a bus 105. In this example the external interface 104 can be used to connect the computer system 100 to the measuring device 110, as well as allowing optional connectivity to other peripheral systems, such as communications networks, databases or other storage devices, or the like. Although a single external interface 104 is shown, this is for the purpose of example only, and in practice multiple interfaces using various methods (eg. Ethernet, serial, USB, wireless or the like) may be provided.

In use, the processor 101 executes instructions in the form of application software stored in the memory 102, to allow different cognitive tasks to be performed, as well as to interpret the signals received from the measuring device 110. Accordingly, it will be appreciated that the computer system 100 may be formed from any suitable processing system, such as a suitably programmed PC, Internet terminal, lap-top, hand-held PC, smart phone, PDA, web server, or the like. Thus, in one example, the computer system 100 is a standard computer system such as a 32-bit or 64-bit Intel Architecture based computer system, that executes software applications stored on non-volatile (e.g., hard disk) storage, although this is not essential.

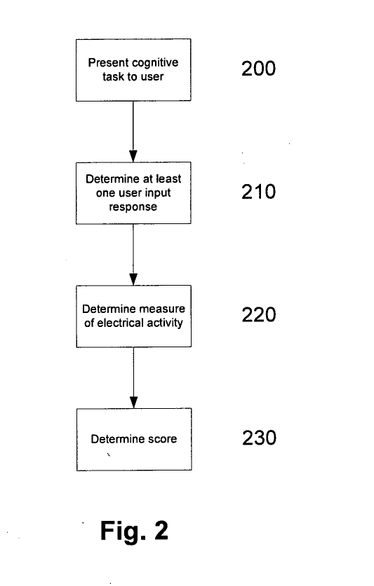

An example of the process for performing cognitive training will now be described with reference to FIG. 2.

In this example, at step 200, the computer system 100 presents a cognitive task to the user. The nature of the cognitive task will depend on a number of factors, such as the nature of the cognitive training being provided, the age and/or mental capacity of the user, or the like.

In one example, the process is used to assist users and in particular children, with neurobehavioural conditions, such as ADHD (Attention Deficit/Hyperactivity Disorder). Such conditions are characterized by impulsiveness and inattention, which render learning difficult. Accordingly, in this example, the cognitive tasks are typically in the form of games that target specific aspects of a user's brain function, such as inhibition and working memory. By focusing on these aspects of brain function, this can help improve the user's inhibition and ability to concentrate, thereby counteracting the effects of the condition and reducing the impact of symptoms.

Inhibition can be targeted through the use of so called go-nogo tasks, on which children with ADHD have consistently been shown to perform more poorly than non-ADHD children, whilst working memory can be addressed by spatial memory tasks, as will be described in more detail below.

It will therefore be appreciated however that different aspects of brain function could be targeted, for example, to assist with different conditions.

Additionally, the techniques can be applied to more general neurological training and does not need to be used in the context of individuals with specific conditions. Thus, for example, the technique could be used to improve attention, memory and inhibition for any user and the reference to neurological conditions is therefore for the purpose of example only.

Similarly, the use of games as cognitive tasks is for the purpose of example only and is not intended to be limiting. Thus, other forms of task could be used, such as puzzles or problem solving, as may be more appropriate for example, for adult users. However, interactive games are particularly useful for children with neurobehavioural conditions as the game encourages use by individuals that often would otherwise find it difficult to participate in a task for any given length of time.

At step 210, whilst the user is performing the cognitive task, the computer system 100 will monitor one or more input responses provided using the input device 103, allowing a measure of the user's performance of the cognitive task to be determined. Thus, in one example above, mistakes in responses made by the user can be used to measure the user's success at performing the task.

At step 220, the computer system 200 determines a measure of electrical brain activity using signals received from the measuring device 110. In one example, the signals are used to generate an indicator of brain electrical activity, which is in turn used to determine a score indicative of an aspect of brain function. In one example, this can be achieved by comparing the indicator to a reference, such as previous measurements made for the user, or from measurements obtained from a sample population of similar individuals, thereby allowing the user's relative attention level to be determined.

At step 230, the computer system 100 determines a score based on any input responses and the measured electrical activity. In one example, the score can be formed from a first score indicative of the user's performance at completing the task, and a second score indicative of the user's attention. The score can then be displayed to the user, allowing the user to determine both their success at performing the task and their level of attention.

Displaying both scores is not essential, and any suitable score can be displayed. However, by providing direct feedback of both scores, this can assist users or other individuals in understanding when the user is attending and how well they are performing with the tasks. This can in turn be used to help the user increase their attention levels. For example, the score can be tied to a reward system, so that rewards are provided in certain conditions, such as if an increase in attention is shown. This encourages participation, and helps improve attention outcomes for the user.

It will be appreciated therefore in this example, the user's brain function is not used to control the performance of the task, which is instead performed in a substantially normal manner. Instead the measure of electrical activity in the user's brain is used to assess how well the user is attending when performing the task. This more closely reflects natural learning when individuals are required to pay attention when performing tasks, such as reading, writing or the like. Thus, by allowing the user to perform the cognitive task using input responses provided via an input device other than the measuring device, this ensures that performance of the cognitive task is similar to performing learning.

In addition to this however, by providing a score indicative of the user's brain function, and in one example, attention, this allows a quantitative assessment to be made as to how well the individual is paying attention. By tying the process into a reward system, the user can be encouraged to pay more attention, with the result of this being directly measurable using the measuring device, thereby assisting the user in managing neurobehavioral conditions.

Accordingly, it will be appreciated that using the measuring device 110 to passively monitor the user's performance at a cognitive task, rather than using the measuring device 110 to control a game or the like, can dramatically improve outcomes for user's with neurobehavioral conditions. Additionally, similar techniques can also be applied to healthy individuals, allowing for general improvement in cognitive function.

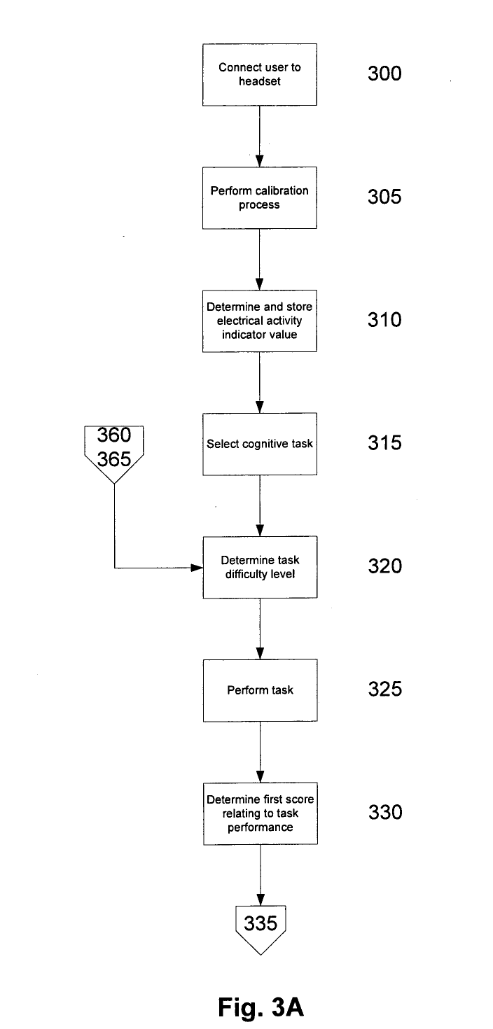

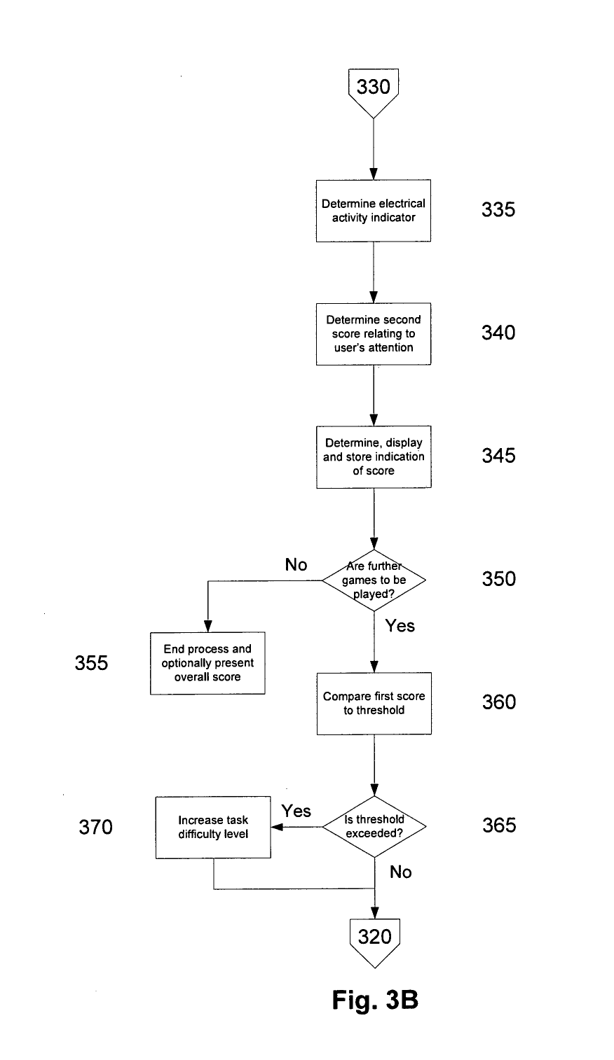

An example process will now be described in more detail with reference to FIGS. 3A and 3B.

In this example, at step 300, the user is connected to a headset incorporating at least the sensing elements of the measuring device. In one example, headset is a NeuroSky™ headset, although this is not essential.

At step 305 a calibration process is performed. The calibration process is used to establish a measure of electrical activity in the user's brain when the user is not attending to a task. Accordingly, this will typically involve having the user sit and stare at a blank screen or other object, with the computer system 100 then using signals from the measuring device to establish a baseline electrical activity indicator, which is then typically stored in memory 102, at step 310.

When a measuring device such as a NeuroSky headset is provided, the electrical activity indicator is based at least in part on parameters generated by the headset, which are at least in part indicative of beta brain wave activity levels in the user's brain. In one particular application, the electrical activity indicator is provided in the form of attention and meditation level values between 0 and 100, derived from the EEG and calculated on-board within the NeuroSky™ headset. Additionally, other EEG information calculated on-board the headset, such as signal quality, EEG band powers (delta, theta, alpha x 2, beta x 2, gamma x 2), or the like, can also be recorded. The additional information could be partially used in determining the electrical activity indicator, or alternatively could be recorded for other purposes, such as to assist with subsequent analysis, or the like.

However, it will be appreciated that any measure indicative of the user's attention level could be used.

At step 315, a cognitive task is selected. In one example, two tasks may be provided for training inhibition and working memory, as described above, in which case the user can select a respective one of the tasks using an appropriate input command provided via in the input device 103.

At step 320, the computer system 100 determines a difficulty level. This can be achieved in any suitable manner, such as by appropriate user input, or by examination of the result of previous tasks, as will be described in more detail below.

At step 325, the task is performed by having the computer system 100 present the task to the user, and monitor user input responses. The user input responses can include not only an input provided by the user, but also the time taken by the user to provide the input, which it will be appreciated can also be indicative of the user's attention level. At step 330, a first score relating to the task performance is determined, with this usually being achieved by comparing the input responses to required responses to identify mistakes by the user, which are in turn used to establish the score. Example tasks will described in more detail below.

At step 335, an electrical activity indicator is determined using signals from the measuring device, with this being used to generate a second score, indicative of the user's attention at step 340. The second score is typically in the form of reward points that are only allocated for attention if (a) the attention level is greater than 25%, and (b) task performance is good or excellent (i.e. equal to or less than one error), encouraging concurrent high levels of general attention and effective use of memory and inhibition.

At step 345, an indication of a score is typically displayed to the user, and stored in the memory 102. In one example, the score can be presented as a combined score. However, in another example, the user is presented with both the first and second scores, allowing the user to establish both their performance in the task and the level of attention. This avoids a user achieving a high score solely by performing well at easy tasks without sufficient attention.

At step 350, it is determined if further games are to be played, and if not the process ends, with scores for the overall session being displayed.

In this regard, in some circumstances, users are required to perform the tasks in sessions, with each session lasting for a set period of time, such as half an hour, or involving a set number of tasks, depending on the requirements of the user. Sessions are repeated daily for a prolonged time period, such as several weeks. Repeating the process in this manner helps reinforce the user's attention during cognitive tasks, making this more natural for the user.

This can be used to reduce the impact of the neurobehavioral conditions where these are present, or can generally improve attention, memory and inhibition for any other users.

Accordingly, if further tasks are to be performed, the computer system 100 compares the first score to a threshold at step 360 and determines if the threshold is exceeded at step 365. If so, then at step 370, the difficulty level is increased. Following this, or otherwise, the process returns to step 320, allowing the next cognitive task to be performed.

By determining the difficulty level using the first score from a previous task, this ensures that the difficulty level is selected based on the ability of the user to perform the task, thereby ensuring that the user is mentally taxed in performing the task. By also displaying the second score however, this allows the user to determine their attention levels. By observing improvements in attention this encourages the user, which in turn leads to further improvements. The user can also be further rewarded externally to the task, providing further encouragement.

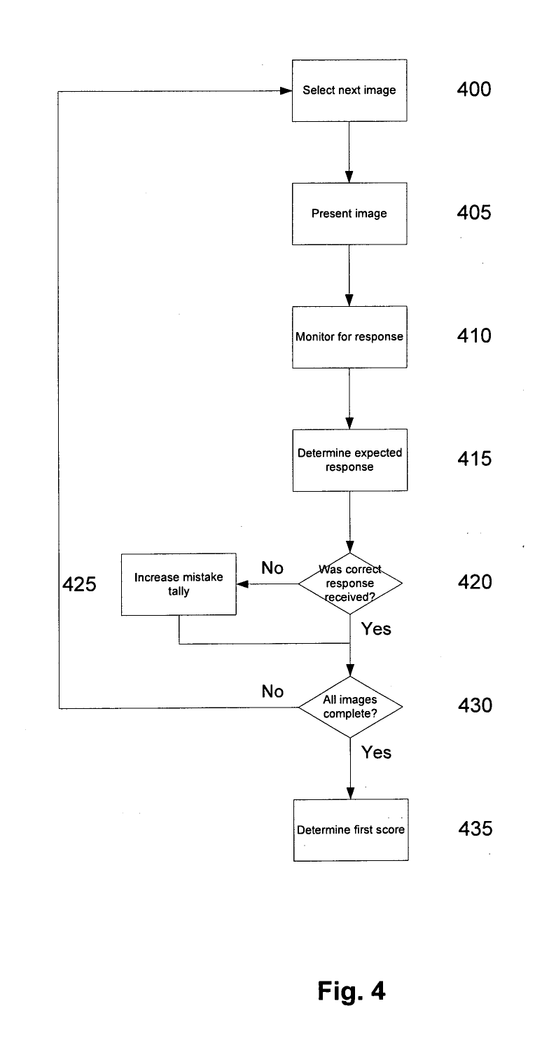

A first example of an inhibition task will now be described with reference to FIG. 4. In this example, the inhibition task involves presenting sequences of images to a user, with the user being required to provide a positive response in response to one particular image category each time it is presented, with a null response being provided for any other images. In this regard, it should be noted that there is evidence that go or nogo responding based on categories of stimuli (e.g. go=fish, nogo=birds), rather than just image A=Go and image B=Nogo, is more generalisable, thereby making the use of categories of greater value.

Accordingly, in this example, at step 400 the computer system 100 selects a next image before presenting this at step 405. At step 410 the computer system monitors for a user input response provided via the input device 103. At step 415 it is determined if a response is expected based on the image presented, with the expected response being compared to any user input response, to determine whether the correct response has been provided, at step 420. If not, then a mistake tally is increased at step 425.

It is then determined if all images have been presented at step 430, and if not, then the process returns to step 400 to select a next image. Otherwise the first score is determined at step 435 based on the mistake tally. Thus, for example, if the mistake tally is zero, a maximum score will be obtained, with the score reducing as the mistake tally increases.

During this process, additional information can be recorded in a data file to allow the information to be used in subsequent analysis of the user task performance. Thus, in one example, in addition to recording the first and second scores, additional information such as response times can be recorded. This information can be used by health professionals to assist in understanding whether the cognitive training is having a positive impact on the user. For example as would be evidenced by a reduction in response times, as response inhibition is relatively more difficult when a fast compared to slower response is required.

A further option is for attention indicators to be displayed to the user based on the attention level of the user. Attention indicators are typically indicators representing the user's attention level as determined based on the electrical activity indicator, and can be used to provide further feedback to the user regarding their attention level. This can be used for example to allow user's to identify if their attention level will be sufficient to earn reward points.

In one example, the attention level indicator could be displayed continuously, providing real time feedback to the user regarding their attention level. Alternatively, however, the attention indicator could be displayed to the user at the end of the game, allowing the user to view their average attention during the game play.

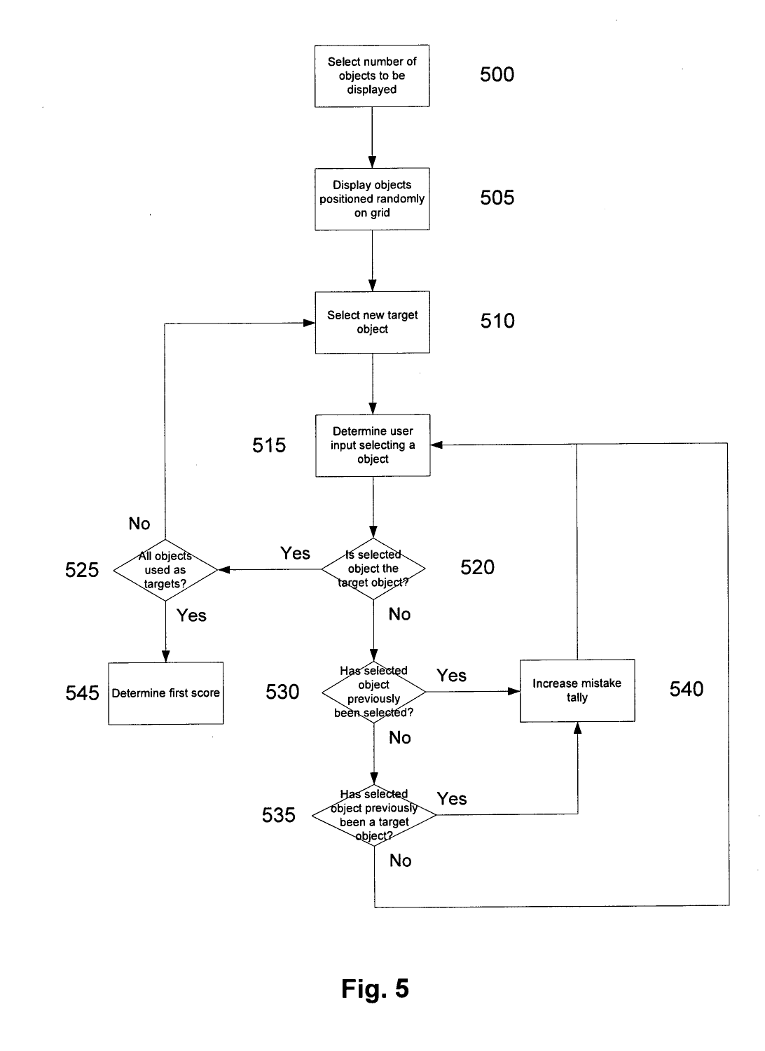

A second example task will now be described with reference to FIG. 5. In this example, objects are presented to the user, with the user being required to randomly select objects to determine a hidden target, for an example an object that is “hiding” an item. The objects are then reset, with the process being repeated but with a different target object.

At step 500, the computer system 100 determines a number of objects to be presented, and displays these randomly on a grid at step 505. At step 510 the computer system 100 selects a target object, before determining a user input representing selection of an object. At step 520, the computer system 100 determines if the selected object is the target object. If so, then at step 525 it is checked if all objects have been target objects, and if not the process returns to step 510 to determine a new target object.

If the selected object is not the target object, then at step 530 it is determined if the object has been previously selected by the user. If not, then it is determined if the selected object has previously been a target object, and if not the process returns to step 515 to allow another object to be selected. Otherwise, at step 540, the mistake tally is increased, meaning that mistakes occur if the user repeatedly checks the same object, or if the user checks an object that has previously been a target object.

Once all objects have been target objects, and successfully identified, then at step 545 the mistake tally is used to determine the first score.

Specific example of such cognitive games aimed at helping children with ADHD will now be described, although it will be appreciated that these or other similar games could also be used to assist in skill development for impulse control (inhibition), working memory and attention, for any individual, and is not intended to be limited for use with children having ADHD.

Go-Go-Nogo (GGNG)

A game of GGNG consists of 32 picture presentations on-screen (each displayed for 0.5 sec), with 21-24 of these being GO pictures (requiring a response from the user, i.e. a mouse click on the Go button) and the remainder being NOGO pictures (no response is required from the user). The Go picture category is displayed on-screen before the start of each new game, e.g. “Cars, 3, 2, 1, go”.

In one example, these events (i.e. button-press' or picture presentations) are recorded in a data-log file with an appropriate code in a given column. Data is written to the data-log file at 1 Hz and a list of the codes for the data-log file are shown in Table 1 below. Raw EEG data can also be logged to an EEG-log file at 128 Hz.

After a response is made to a Go picture, or no response is made to the Nogo picture, there is a delay until the next picture is presented, called the inter-stimulus-interval (ISI). The ISI varies according to the difficulty level of the game, by reducing with increasing levels. At level 1 the ISI is 2.0 sec; level 2, 1.8 sec; level 3, 1.6 sec; level 4, 1.4 sec; level 5, 1.2 sec; level 6, 1.0 sec; level 7 0.8 sec; level 8, 0.6 sec; level 9, 0.4 sec; level 10, 0.2 sec.).

At the end of each game reward points (RP) are determined based on performance in that game. If there were no errors, difficulty level increases by 1, and the user is allocated 10 RP. If one error was made, difficulty level remains unchanged and 5 RP are allocated. At level 1, if >1 error is made, difficulty remains at level 1, and no RP are allocated. From level 2 onwards, if >1 error is made, difficulty level reduces by 1, and no RP are allocated. These specifications are the same for GGNG and FTM.

At the end of each game a results screen appears, and provides a brief report on performance, difficulty level variation and RP allocation.

An accumulating tally of RP is kept across the 6 games of GGNG that is required for a training session. This tally is tracked across training sessions as an objective index of performance.

Feed The Monkey (FTM)

Generally, FTM is a search/memory task, and involves searching sets of crates/boxes to find banana's.

A game of FTM consists of a number of boxes being randomly allocated to any locations on a 10×10 grid, one of which is the Target box (contains banana) and the others are Non-targets (empty). The user searches the boxes with the computer mouse and after a Target is located, it is put into another of the search boxes. In between these events text on-screen says “You find them, I hide them!”. Searching then continues until a Target has been located in each of the search boxes for that game. After the final banana is found the game is over.

The number of search boxes varies depending on the difficulty level. At level 1 there are 4 boxes. Level 2 has 5 boxes, up to level 10 with 13 boxes. There is always only one Target box.

In one example, these events (i.e. mouse button-press') are recorded in a data-log file with an appropriate code and in a given column. Data is typically written to the data-log file at 1 Hz and a list of the codes for the data-log file are shown in Table 1 below. Raw EEG data is also typically logged to an EEG-log file at 128 Hz.

A report screen is displayed at the end of each game. The same RP allocation system as for GGNG allocates RPs.

Six games will be played in a training session. A accumulating tally of RP is kept across the 6 games of FTM.

TABLE 1

| Game | Stimulus | Code | Response | Code |
|---|---|---|---|---|
| GGNG | Go | 10 | Correct press | 15 |
| | Nogo | 11 | Missed press | 16 |
| | | | Press to Nogo | 17 |
| FTM | Boxes on screen | 20 | Found Target | 21 |
| | New game | 25 | Found Non-target | 22 |
| | | | Non-target 2^{nd} search | 23 |
| | | | Target 2^{nd} search | 24 |

Attention Meters

The Attention meters operate during game play for both GGNG and FTM. One meter shows Attention level (a number between 0 and 100) on a second-by-second basis, while the other meter shows the accumulating average (0-100) across the current game. These meters are hidden as a default - training on GGNG and FTM occurs with them operating in the background only.

At the end of each game the averaged Attention level is displayed on the results screen along with task performance information, and consequent RP allocation information. An Attention meter average of 0-25 results in no RP, 26-50=10 RP, 51-75=15 RP and 76-100=20 R

Reward points are only allocated for Attention if (a) the Attention level is greater than 25%, and (b) task performance is good or excellent (i.e. equal to or less than one error), encouraging concurrent high levels of general attention and effective use of memory and inhibition.

Experimental Data

Research relating to the use of the above described techniques for optimizing attention, impulse control and working memory functioning in children with and without Attention-Deficit Hyperactivity Disorder (ADHD) will now be described.

The experiment aims to compare a computer-based cognitive training program for optimizing attention, impulse control and working memory functioning (Cog-TS) with the same program when utilised with an attention-focused neuro-feedback component (NeuroCog-TS). Both will be compared to a wait-listed control group.

The study evaluates the efficacy of:
a) a cognitive training software application consisting of two tasks performed on a PC. This condition is termed Cog-TS.
b) the same cognitive training software application as described in (a), with additional attention-focused EEG input via the NeuroSky MindSet™ (a portable, wireless, single channel, dry sensor EEG measurement device). This condition is termed NeuroCog-TS.

Both are compared to a wait-listed control group.

Of the two tasks performed the first task is a response inhibition task, based on the go-nogo paradigm, on which children with ADHD have consistently been shown to perform more poorly than non-ADHD children. The second task is a spatial working memory task on which ADHD children have also consistently shown deficits. These tasks provide children with the opportunity to exercise impulse control and memory processes in the context of more complicated cognitive tasks. For both training groups, the tasks will adjust their difficulty level according to the child's ongoing performance, getting more difficult with error-free performance, with a performance-based reward point system.

Half of the participants are children aged between 7 and 14 years diagnosed with ADHD. The other half are children of the same age without ADHD or any other difficulties. Participants are recruited from a variety of sources using ethically approved methods.

Participants undergo a pre-training assessment session, including assessment of:
(a) Overt behaviour (Connors Parent Rating Scale; purpose-designed DSM-IV ADHD symptom frequency scale)
(b) General cognitive ability (WASI and South Australian Spelling test)
(c) Attention processing (visual oddball task)
(d) Memory processing (digit-span and counting-span tasks)
(e) Impulse control (visual go-nogo and flanker tasks)
(f) Brain electrical activity “state” (eyes-open and eyes-closed EEG)

These take about 1.5 hours to complete. The participants are randomly allocated to either the Cog-TS, NeuroCog-TS or wait-listed group. The participants are typically required to complete a predetermined number of sessions, such as 25 sessions, which in one example is achieved by having the user engage in the tasks for approximately 20 minutes, 5-6 times a week for 4 weeks in their home. Children play 6 ‘games’ each of the inhibition and working memory tasks per training session. Parents of the children are contacted by phone every two weeks to encourage compliance with the intervention and the computer program keep a log file recording when the program was used and parameters of the task. Pre- and post-testing sessions are performed as required. After 4 weeks participants complete a post-training assessment session, in which participants complete the same tasks as at the pre-training session, allowing an assessment of any improvement to be performed. Data log files generated by the software can be further used in assessing the effectiveness of the process.

In one example, results of each training session are stored in the memory 102, allowing the results of previous sessions to be displayed to the user each time a new training session is commenced. At the end of the 25 training sessions the total number of Reward Points earned is compared to a 3-level hierarchy of achievement, with linked rewards (termed the Training Goals) established prior to training commencement. The appropriate reward is provided based on either level 1, level 2 or level 3 achievement. In general, testing demonstrates that the above described cognitive tasks lead to one or more general effects (NeuroCog-TS>Cog-Ts>Wait list group) including:
1. Improvements in overt behaviour, as indicated by the Connors Parent Rating Scale and purpose-designed DSM-IV ADHD symptom frequency scale.
2. Improvements in general cognitive ability, as indicated WASI and South Australian Spelling test.
3. Improvements in attention processing, as indicated by brain electrical activity “state” measures, and the visual oddball task post-training.
4. Improvements in memory processing, as indicated by FTM in the training context, and as generalised to digit-span and counting-span tasks post-training.
5. Improvements in impulse control, as indicated by GGNG in the training context and as generalised to the visual Go-Nogo and Flanker tasks post-training.

Persons skilled in the art will appreciate that numerous variations and modifications will become apparent. All such variations and modifications which become apparent to persons skilled in the art, should be considered to fall within the spirit and scope that the invention broadly appearing before described.

Thus, for example, whilst the above described process has been described for use in assisting user with neurobehavioural conditions or disorders such as ADHD, the technique can also be used to assist brain function such as attention in healthy individuals.

Additionally, by establishing a database of reference scores and response times for individuals with conditions, this can also be used to assist in establishing the need to diagnose conditions such as ADHD, or the like.

1. A method of providing cognitive training to a user, the method including, in a processing system:
a) presenting a cognitive task to a user, the cognitive task requiring the user to view presented information and provide at least one input response;
b) determining the at least one input response using an input device;
c) determining from a measuring device a measure of electrical activity in the user's brain whilst performing the cognitive task; and,
d) determining a score based on at least one of the at least one input response and the measured electrical activity.

2. A method according to claim 1, wherein the cognitive task includes a number of difficulty levels, and wherein the method includes, in the processing system, selecting a difficulty level for the task at least partially in accordance with the score from a previous cognitive task.

3. A method according to claim 2, wherein the method includes, in the processing system, selecting a difficulty level for the cognitive task in accordance with a score indicative of a success measure of a previous cognitive task.

4. A method according to claim 1, wherein the method includes, in the processing system:
a) determining a first score using the at least one input response;
b) determining a second score using the measured electrical activity; and,
c) determining the score using the first and second scores.

5. A method according to claim 4, wherein the method includes, in the processing system:
a) comparing the input response to a required response; and,
b) determining the first score in accordance with results of the comparison.

6. A method according to claim 1, wherein the method includes, in the processing system:
a) determining an electrical activity indicator using the measured electrical activity;
b) comparing the electrical activity indicator to indicator criteria; and,
c) determining a second score in accordance with results of the comparison.

7. A method according to claim 6, wherein the electrical activity indicator is indicative of at least one characteristic of brain function.

8. A method according to claim 7, wherein the electrical activity indicator is at least partially indicative of at least one of:
a) attention; and,
b) focus.

9. A method according to claim 6, wherein the method includes, in the processing system:
a) determining a baseline electrical activity indicator using the measured electrical activity prior to performing a cognitive task; and,
b) determining indicator criteria at least partially in accordance with the baseline electrical activity indicator.

10. A method according to claim 1, wherein the task is at least one of:
a) an inhibition task;
b) a memory task; and,

11. A method according to claim 1, wherein the method includes, in the processing system:
a) presenting a sequence of representations to the user, each representation being associated with a respective required response;
b) for each representation, comparing at least one input response to the respective required response; and,
c) determining a score at least partially in accordance with results of the comparison.

12. A method according to claim 11, wherein the at least one required response associated with at least one of the representations is a null response.

13. A method according to claim 1, wherein the method includes, in the processing system:
a) selecting one of a plurality of objects to be a target object;
b) presenting representations of the plurality of objects to the user;
c) determining user selection of an object in accordance with an input response;
d) determining if the user selected object is the target object; and,
e) determining a score at least partially in accordance with results of the determination.

14. A method according to claim 1, wherein the score is used to assess cognitive function.

15. A method according to claim 1, wherein the score is used as an incentive to improve cognitive function.

16. A method according to claim 1, wherein the method is used in the treatment of neurobehavioural conditions.

17. An apparatus for providing cognitive training to a user, the apparatus including a processing system for:
a) presenting a cognitive task to a user, the cognitive task requiring the user to view presented information and provide at least one input response via an input device;
b) receiving from a measuring device a measure of electrical activity in the user's brain whilst performing the cognitive task; and,
c) determining a score based on at least one of the at least one input response and the measured electrical activity.

18. The apparatus according to claim 17, wherein the processing system includes:
a) a memory for storing instructions; and,
b) a processor for executing the instructions to thereby:
i) presenting the cognitive task to the user;
ii) determine the at least one input response;
iii) receive the measure of electrical activity; and,
iv) determine the score.

19. The apparatus according to claim 18, wherein the processing system includes the input device.

20. The apparatus according to claim 17, wherein the apparatus includes the measuring device.
